(19) United States
(12) Patent Application Publication
(10) Pub. No.: US 2010/0167400 A1
(43) Pub. Date: Jul. 1, 2010

(54) Composition for Increasing Intracellular Nitric Oxide and Method for the Same

(76) Inventors: Ting-Kai Leung, Taipei City (TW); Yuan-Soon Ho, Taipei City (TW); Huey-Fang Shang, Taipei City (TW); Yi-Hsuan Lee, Taipei County (TW); Yung-Sheng Lin, Changhua County (TW); Ming-Yu Lin, Hsinchu City (TW); Jiann-Shiun Kao, Taipei City (TW)

Correspondence Address: HAVERSTOCK & OWENS LLP, 162 N WOLFE ROAD, SUNNYVALE, CA, 94086, US

(21) Appl. No.: 12/087,072

(22) PCT Filed: Jan. 1, 2007
(86) PCT No.: PCT/US2007/012977

§ 371 Date: Jun. 20, 2008

(51) Int. Cl. C12N 5/078; C12N 5/00; C12N 5/09; C12N 5/079; C01F 7/02

(52) U.S. Cl. 435/375; 423/625

(57) ABSTRACT

A composition and a method for increasing intracellular nitric oxide is provided. The composition comprises a far-infrared ray releasing substance composed mainly of an oxide mineral for releasing a far-infrared ray, wherein the composition promotes generation of the nitric oxide via irradiation of the far-infrared ray from the far-infrared ray releasing substance. In another aspect, the method comprises setting an effective amount of a far-infrared ray releasing substance in a place close to a cell with an appropriate distance, and incubating the far-infrared ray releasing substance with the cell for a specific period, wherein the appropriate distance lies in an irradiation range of the far-infrared ray releasing substance.

### FIELD OF THE INVENTION

The present invention relates to a composition and a method for increasing intracellular nitric oxide through far-infrared ray irradiation by using mineral oxides.

### BACKGROUND OF THE INVENTION

Nitric oxide is a free radical gas, and an important regulator in our body. It can regulate the micro-circulation by mediating endothelium-dependent vaso-dilation. Moreover, nitric oxide is also a neurotransmitter, that mediate intraneural signaling for neuronal survival and neuroplasticity. However, nitric oxide serves as an attacker in immune cells, which helps immune cells eliminate microorganism and cancer cells. Therefore, producing and maintaining nitric oxide at the physiological level is very important in the cardiovascular system, the nervous system and immune system.

According to International Commission on Illumination (CIE1987), the far-infrared ray (FIR) is an electromagnetic wave with the wavelength of 3-1000 μm. Among them, the far-infrared ray having the wavelength of 3-14 μm is called the light of life, because of its advantages in the growth of animals and plants. Currently, it has been proved that FIR has therapeutic effect on many human diseases, and thus is often applied on many physiological purposes, for example, blood circulation acceleration, metabolism activation, tissue regeneration and immune system activation, etc. FIR brings both thermal and non-thermal effects, wherein the thermal effect includes a slight elevation of the regional tissue temperature, and the non-thermal effect includes influence on cell functions such as cell proliferation and promotion of immune cell functions.

Currently, most of the prior studies use emitting sources of FIR powered by electricity; nevertheless, they cannot be performed without the aid of the outside heat source and are not easy to carry. Furthermore, some FIR sources contain excess rare elements, which result in radioactive irradiation when used. However, the present invention provides a composition that promotes intracellular nitric oxide production by releasing FIR under room temperature, and it has the effect on cell physiology. Although the composition in the present invention is a radioactive substance, it will not release free irradiation, and it also has negative ion that is beneficial to the human body. Hence, it is guaranteed that the users will be safe and healthy.

In view of the drawbacks of current techniques, the inventors develop a composition that releases FIR through the non-thermal effect thereof, and such non-thermal effect enhances intracellular nitric oxide. The present invention provides a composition and a method for increasing intracellular nitric oxide. The summary of the present invention is described below.

### SUMMARY OF THE INVENTION

It is an aspect of the present invention to provide a composition for increasing nitric oxide in a cell comprising a far-infrared ray (FIR) releasing substance which promotes generation of the nitric oxide via irradiation of the far-infrared ray therefrom. In specific embodiments of this invention, the FIR releasing substance can promote nitric oxide production in cancer cells, antigen presenting cells and neural cells at room temperature. In a preferred embodiment, the FIR releasing substance is ceramic powder and composed of 60-95% aluminum in weight.

It is another aspect of the present invention to provide a pharmaceutical composition for increasing nitric oxide comprising a pharmaceutically effective amount of a far-infrared ray releasing substance. In specific embodiments, this pharmaceutical composition can be used to treat nitric oxide-defective diseases such as cancer, immune deficiency diseases and neural degenerating diseases.

It is a further aspect of the present invention to provide a method for increasing nitric oxide. In an exemplary embodiment, the FIR releasing substance increases the nitric oxide generation in a cell without directly contacting with the cell for a specific period, for instance, being placed beneath the cell cultured dish for 10-60 minutes.

Other objects, advantages and efficacies of the present invention will be described in detail below taken from the preferred embodiments with reference to the accompanying drawings, in which:

### BRIEF DESCRIPTION OF THE DRAWINGS

FIG. 1 is a bar graph showing the effect of the FIR releasing substance of the present invention on the amount of nitric oxide in a cancer cell (MCF-7), and the solid bars herein represent control groups whereas the empty bars represent groups of FIR releasing substance, and the incubating periods are 0 minute, 10 minutes and 60 minutes, respectively, and 10-90 and 60-90 stand for removing the FIR releasing substance for 90 minutes after a 10 or 60 minutes incubation;

FIG. 2 is a bar graph showing the effect of the FIR releasing substance of the present invention on the amount of nitric oxide in an antigen presenting cell, wherein M represents the cells cultured in medium, and LPS represents the cells cultured in medium containing 600 ng/mL lipopolysaccharide (LPS), and the black bars represent the control group whereas the gray bars represent groups of FIR releasing substance;

FIG. 3 is a bar graph showing the effect of the FIR releasing substance of the present invention on the amount of nitric oxide in neural cells, wherein the ctrl stands for the control group, the FIR stands for FIR groups, and the result is indicated with average fluorescent intensity;

FIG. 4 is a diagram showing the measuring result of the amount of free irradiation of the FIR releasing substance in the present invention; and

FIG. 5 is a diagram showing the measuring result of the amount of negative ion of the FIR releasing substance in the present invention.

### DETAILED DESCRIPTION OF THE PREFERRED EMBODIMENT

### I. Definitions

The following definitions are provided in order to aid an understanding of the detailed description of the present invention:

The term “antigen presenting cells” as used herein refers to immune cells having phagocytic ability, and can activate T or B cells consequently. Generally, the antigen presenting cells includes macrophages and dendritic cells.

The term “nitric oxide-defective disease” as used herein refers to the diseases resulting from nitric oxide deficiency, including but not limited in hypertension, cardiovascular diseases, diabetes, cancer, neural degenerating disease and immune deficiency diseases. In one embodiment, the nitric oxide-defective disease is cancer.

In the present invention, the term “without directly contacting” refers to that the FIR releasing substance is not directly added into the cell cultured medium, and thus does not affect growth of the cells. In one embodiment, the FIR releasing substance is placed beneath the cell cultured dishes.

### II. DETAILED DESCRIPTION

### Example I

### The Compose of the Far-Infrared Ray (FIR) Releasing Substance

Preferably, the FIR releasing substance of the present invention is ceramic powder of micro-sized particles that include 60-95% aluminum oxide in weight. Besides the aluminum oxide, the FIR releasing substance may include other ingredients such as titanium dioxide, titanium boride, magnesium oxide, silicon oxide, iron oxide, zinc hydroxide, zinc oxide and carbides, etc. Besides powders, the FIR releasing substance is also in form of a bulk, a grain or a membrane. The average emissivity of the ceramic powder is 0.92 and over 0.98 at a wavelength between 4-14 μm and 6-14 μm, respectively. As Table. 1 shows, the result represents an extremely high ratio of far-infrared ray intensity.

TABLE 1

| Wavelength | FIR emissivity |
|---|---|
| 4~14 μm | 0.92 |
| 6~14 μm | 0.98 |

### Example II

### The Effect of the FIR Releasing Substance on Increasing Nitric Oxide in MCF-7 Cell

Equal amount of 100 gm FIR powder (FIR groups) and nonfunctional milk powder (control groups) were enclosed by different bags, which are made of synthetic or natural high polymer, metal, glass or ceramics, etc.

MCF-7 Cells Culture

The human breast cancer cell line MCF-7 was grown in suspension in the MEM medium with 10% fetal calf serum supplemented with 1 mM sodium pyruvate, and incubated (37° C., 5% CO₂) in the dark. The bags filled with FIR powder (as FIR groups) and bags filled with non-functional powder (as control group) were inserted beneath the dishes of MCF-7 cells, which are irradiated by FIR ceramic powder without direct contact. The dishes with placing FIR powder (as FIR group) and nonfunctional powder (as control group) were divided into five categories: (1˜3), placed for 0 minute, 10 minutes and 60 minutes intervals with treatments of powder-bags; (4˜5), placed for 10 minutes and 60 minutes respectively and then taken away from the powder-bags until 90 minutes.

Flow Cytometry Measurement

All the dishes were followed by the staining of DAF-FM diacetate for fluorescence and measurement. The DAF-FM diacetate can penetrate cell membrane, and is used for labeling the nitric oxide synthase and determining the activity thereof in the cell. All the cells were analyzed by fluorescence-activated cell sorter (FACS) and flow cytometry at the single-cell level. As the data were acquired and analysis, the mean fluorescence intensities of the breast cells were determined in comparison with the control groups.

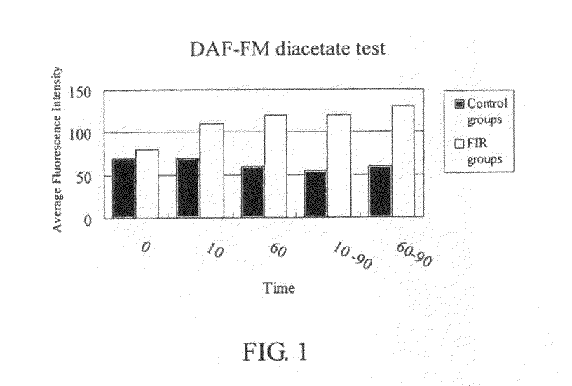

Please refer to Table. 2 and FIG. 1, which illustrates the result of nitric oxide production of the second embodiment of the present invention. In FIG. 1, the solid bars represent control groups whereas the empty bars represent groups of FIR releasing substance. The incubating periods are 0 minute, 10 minutes and 60 minutes, respectively, and 10-90 and 60-90 stand for removing the FIR releasing substance for 90 minutes after a 10 or 60 minutes incubation. As shown in Table. 2 and FIG. 1, a significant difference exists between these two groups, and the result demonstrates that FIR releasing substance of the present invention could induce the nitric oxide (NO) synthesis in breast cancer. In addition, there were 37.5% increase of NO generation after 10 minutes and 50.0% increase after 60 minutes of FIR irradiation. There were further increase to 50.0% and 62.5% by 90 minutes after the completeness of 10 minutes- and 60 minutes-FIR irradiation respectively (post FIR effect). Based on the above results, it is proved that the FIR releasing substance of the present invention can increase intracellular nitric oxide effectively.

TABLE 2

Groups

| Time | Control | FIR (cumulative increase % of NO) |
|---|---|---|
| 0 | 70 | 80 (0%) |
| 10 | 70 | 110 (37.5%) |
| 60 | 60 | 120 (50%) |
| 10 min then 90 | 55 | 120 (50%) |
| 60 min then 90 | 60 | 130 (62.5%) |
| P value | | 6.63 × 10⁻⁴ |

### Example III

### The Effect of the FIR Releasing Substance on Increasing Nitric Oxide in RAW 264.7 Cells

RAW 264.7 Cells Culture

RAW 264.7 cells were cultured in DMEM supplemented with 10% fetal calf serum, 10000 I.U./mL penicillin, 10000 μg/mL streptomycin, 25 μg/mL amphotericin, and 1% L-glutamate. The cell number was adjusted to 4×10⁵ cells/mL. Cell suspension (1 mL) were seeded onto a 24-well microtiter plate and LPS (600 ng/mL) were added. After incubating the cells at 37° C. under 5% CO₂ in air for 24 hours, the cultured plate was centrifuged at 1500 rpm for 5 min. The supernatants were collected for NO.

Measurement of NO Production

The cultures supernatant of 100 microliter were added onto a 96-well micotiter plate. 100 μl of Griess reagent (Fluka) were added to each well and placed for 15 minutes at room temperature. The sodium nitrite (0-500 μM) was used as standard. Absorbance was measured at 530 nm and the result was shown as nitrite concentration (μM).

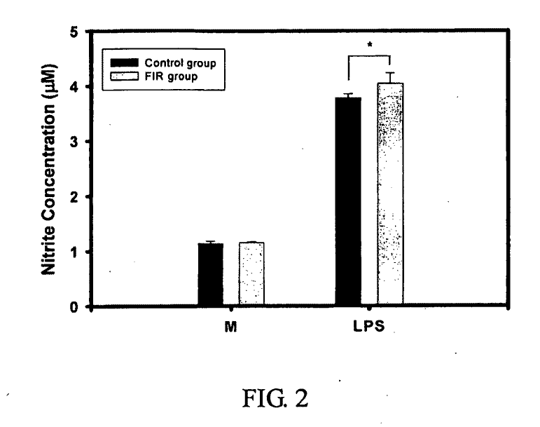

As shown in FIG. 2, M represents RAW 264.7 cells cultured in DMEM medium, and LPS represents RAW 264.7 cells cultured in DMEM medium supplemented with 600 ng/mL LPS. The black bars represent the control group without FIR releasing substance placement, whereas the gray bars represent FIR groups with FIR releasing substance beneath the culture dishes. LPS is an activator of macrophages, which induces nitric oxide production by nitric oxide synthase in the cells. Therefore, the nitrite concentration of LPS co-cultured cells is higher than that of medium-only cultured cells.

It is noticed that whether culturing with LPS or not, the nitrite concentration of FIR groups is higher than that of the control groups, showing that the FIR releasing substance constructs an environment for enhancing nitric oxide production in RAW 264.7 cells. The slight enhancement of nitrite concentration in cells is due to the non-thermal effect of the FIR releasing substance. Accordingly, it is demonstrated that the FIR releasing substance has an effect of promoting nitric oxide production in such antigen presenting cells.

### Example IV

### The Effect of The FIR Releasing Substance on Increasing Nitric Oxide in Astrocytes

Astrocytes Culture

1-2-day-old neonatal Sprague-Dawley rats were anesthetized and sacrificed by an overdose of sevoflurane. Rat brains were then harvested and homogenized by mechanical dissociation. The cell suspensions were diluted with DMEM/F12 supplemented with 10% heat-inactivated fetal calf serum and 100 U/ml penicillin-streptomycin sulfate (Invitrogen, Carlsbad, Calif.). Cells were seeded onto 75-cm² flasks at an initial density of 2×10⁶ cells per flask. Astrocytes were cultured to confluence in a 5% CO₂ incubator at 37° C. Upon confluency, cells were dissociated using 0.25% trypsin/0.02% EDTA (Invitrogen, Carlsbad, Calif.), washed and subcultured onto 6-cm dishes, cultured to confluency, and used for experiments.

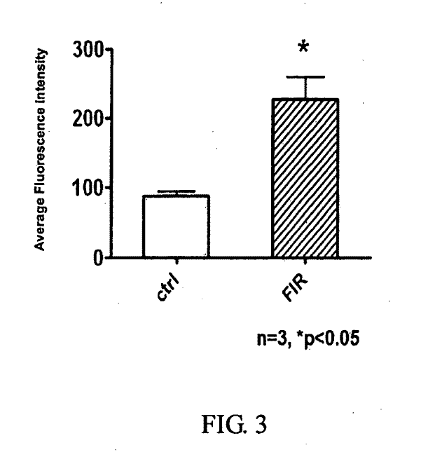

Please refer to FIG. 3, which is a bar graph showing the effect of the FIR releasing substance on increasing nitric oxide in astrocytes. The ctrl stands for the control group, and the FIR stands for FIR groups. The procedures of flow cytometry measurement are described as above, and the result is indicated with average fluorescent intensity. As FIG. 3 shows, the FIR releasing substance can enhance the amount of nitric oxide in astrocytes for about 2 folds. Therefore, the FIR releasing substance has the advantage of promoting nitric oxide production in neural cells.

### Example V

### Free Irradiation Measurement

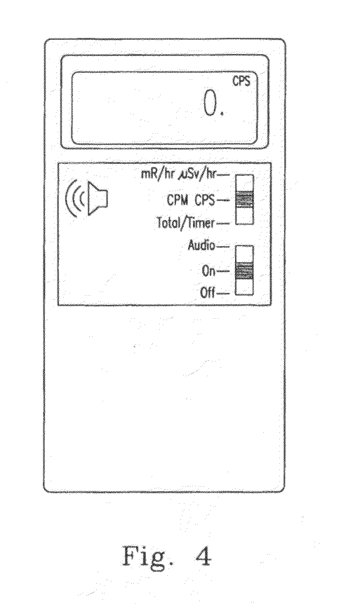

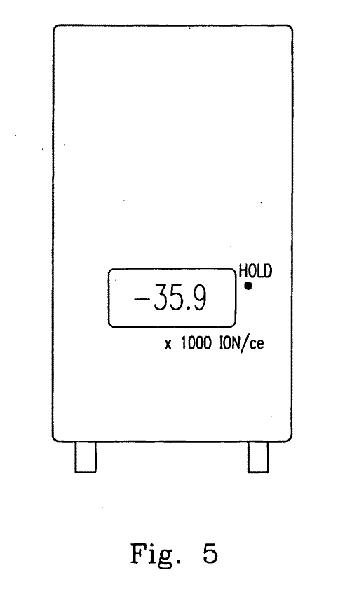

The amount of free irradiation of the FIR releasing substance of the present invention was measured by an irradiation measuring instrument. FIG. 4 illustrates that the amount of free irradiation of the FIR releasing substance is 0. According to the measuring result, the FIR releasing substance does not have harmful free irradiation, although it is a radioactive substance. Additionally, the amount of negative ions released by the FIR releasing substance of the present invention was measured by a negative ions measuring instrument. As FIG. 5 shows, the amount of negative ions per unit reaches 35,900 ions.

To summarize, the present invention proposes a composition and a method for increasing nitric oxide in cells, wherein the composition promotes generation of the nitric oxide via an irradiation of the far-infrared ray from the far-infrared ray releasing substance at room temperature, thereby improving the drawbacks of the prior art. Further, the composition regulates biological effects of the neural system, cardiovascular system and immune system through nitric oxide production. Thus, the present invention not only bears novelty and obviously progressive nature, but also bears the utility for the industry.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

1. A composition for increasing a nitric oxide in a cell, comprising:
a far-infrared ray releasing substance composed mainly of an oxide mineral for releasing a far-infrared ray;
wherein the composition promotes a generation of the nitric oxide via an irradiation of the far-infrared ray from the far-infrared ray releasing substance.

2. The composition as claimed in claim 1, wherein the cell is one selected from a group consisting of a cancer cell, a neural cell, an endothelium cell and an antigen presenting cell.

3. The composition as claimed in claim 1, wherein the far-infrared ray releasing substance is in a form selected from a group consisting of a bulk solid, a grain, a powder and a membrane, and releases the far-infrared ray under a room temperature.

4. The composition as claimed in claim 1, wherein the oxide mineral is an aluminum oxide.

5. The composition as claimed in claim 4, wherein the aluminum oxide is 60-95% in weight.

6. A pharmaceutical composition for increasing a nitric oxide, comprising:
a pharmaceutically effective amount of a-far infrared ray releasing substance;
wherein the pharmaceutical composition promotes a generation of the nitric oxide via an irradiation of the far-infrared ray releasing substance.

7. The pharmaceutical composition as claimed in claim 6 being used for improving a nitric oxide-defective disease.

8. The pharmaceutical composition as claimed in claim 6, wherein the pharmaceutically effective amount of the far-infrared ray releasing substance increases the nitric oxide generation in a cell.

9. The pharmaceutical composition as claimed in claim 8, wherein the cell is one selected from a group consisting of a cancer cell, a neural cell, an endothelium cell and an antigen presenting cell.

10. The pharmaceutical composition as claimed in claim 6, wherein the pharmaceutically effective amount of the far-infrared ray releasing substance is in a form selected from a group consisting of a bulk solid, a grain, a powder and a membrane, and releases the far-infrared ray under a room temperature.

11. The pharmaceutical composition as claimed in claim 6, wherein the far-infrared ray releasing substance is composed mainly of an oxide mineral, and the oxide mineral is an aluminum oxide.

12. The pharmaceutical composition as claimed in claim 11, wherein the aluminum oxide is 60-95% in weight.

13. A method for increasing a nitric oxide, comprising:
setting an effective amount of a far-infrared ray releasing substance in a place close to a cell with an appropriate distance, wherein the appropriate distance lies in an irradiation range of the far-infrared ray releasing substance; and
incubating the far-infrared ray releasing substance with the cell for a specific period.

14. The method as claimed in claim 13, wherein the effective amount of the far-infrared ray releasing substance is composed mainly of an oxide mineral.

15. The method as claimed in claim 14, wherein the oxide mineral is an aluminum oxide.

16. The method as claimed in claim 15, wherein the aluminum oxide is 60-95% in weight.

17. The method as claimed in claim 13, wherein the effective amount of the far-infrared ray releasing substance increases the nitric oxide generation in a cell without directly contacting with the cell.

18. The method as claimed in claim 13, wherein the cell is one selected from a group consisting of a cancer cell, a neural cell, an endothelium cell and an antigen presenting cell.

19. The method as claimed in claim 13, wherein the pharmaceutically effective amount of the far-infrared ray releasing substance is in a form selected from a group consisting of a bulk solid, a grain, a powder and a membrane, and releases the far-infrared ray under a room temperature.

20. The method as claimed in claim 13, wherein the specific period is 10-60 minutes.
